(19) United States
(12) Patent Application Publication
(10) Pub. No.: US 2008/0312311 A1
(43) Pub. Date: Dec. 18, 2008

(54) Crystalline forms of 3-(2R-tetrahydrofuryl-methyl)-2-thioxanthine

(75) Inventors: Bengt Leonard Aslund, Sodertalje (SE); Robert Ehrl, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/137,583

(22) Filed: Jun. 12, 2008

Related U.S. Application Data
(60) Provisional application No. 60/943,671, filed on Jun. 13, 2007.

(51) Int. Cl. A61K 31/382; C07D 335/12; A61P 37/02; A61P 25/00; A61P 11/00; A61P 9/00

(52) U.S. Cl. 514/437; 549/26

(57) ABSTRACT

The present invention relates to novel crystalline forms of 3-(2R-tetrahydrofuryl-methyl)-2-thioxanthine. Further the present invention also relates to compositions comprising them and their use in therapy.

### CROSS-REFERENCE TO RELATED APPLICATIONS

This application claims the benefit of United States Provisional Application No. 60/943,671, filed Jun. 13, 2008, the contents of which are hereby incorporated herein by reference.

The present invention relates to novel crystalline forms of 3-(2R-tetrahydrofuryl-methyl)-2-thioxanthine. Further the present invention also relates to compositions comprising them and their use in therapy.

In the formulation of drug compositions, it is important for the drug substance to be in a form in which it can be conveniently handled and processed. This is of importance, not only from the point of view of obtaining a commercially viable manufacturing process, but also from the point of view of subsequent manufacture of pharmaceutical formulations comprising the active compound.

Further, in the manufacture of oral drug compositions, it is important that a reliable, reproducible and constant plasma concentration profile of drug is provided following administration to a patient.

Chemical stability, solid-state stability, and “shelf life” of the active ingredients are also very important factors. The drug substance, and compositions containing it, should be capable of being effectively stored over appreciable periods of time, without exhibiting a significant change in the physico-chemical characteristics of the active component, e.g. its chemical composition, density, hygroscopicity and solubility.

Amorphous materials may present problems in this regard. For example, such materials are typically more difficult to handle and to formulate, provide for unreliable solubility, and are often found to be more unstable.

Thus, in the manufacture of commercially viable and pharmaceutically acceptable drug compositions, it is important, wherever possible, to provide the drug in a substantially crystalline and stable form.

Myeloperoxidase (MPO) is a heme-containing enzyme found predominantly in polymorphonuclear leukocytes (PMNs). MPO is one member of a diverse protein family of mammalian peroxidases that also includes eosinophil peroxidase, thyroid peroxidase, salivary peroxidase, lactoperoxidase, prostaglandin H synthase, and others. The mature enzyme is a dimer of identical halves. Each half molecule contains a covalently bound heme that exhibits unusual spectral properties responsible for the characteristic green colour of MPO. Cleavage of the disulphide bridge linking the two halves of MPO yields the hemi-enzyme that exhibits spectral and catalytic properties indistinguishable from those of the intact enzyme. The enzyme uses hydrogen peroxide to oxidize chloride to hypochlorous acid. Other halides and pseudohalides (like thiocyanate) are also physiological substrates to MPO.

PMNs are of particular importance for combating infections. These cells contain MPO, with well-documented microbicidal action. PMNs act non-specifically by phagocytosis to engulf microorganisms, incorporate them into vacuoles, termed phagosomes, which fuse with granules containing myeloperoxidase to form phagolysosomes. In phagolysosomes the enzymatic activity of the myeloperoxidase leads to the formation of hypochlorous acid, a potent bactericidal compound. Hypochlorous acid is oxidizing in itself, and reacts most avidly with thiols and thioethers, but also converts amines into chloramines, and chlorinates aromatic amino acids. Macrophages are large phagocytic cells, which, like PMNs, are capable of phagocytosing microorganisms. Macrophages can generate hydrogen peroxide and upon activation also produce myeloperoxidase. MPO and hydrogen peroxide can also be released to the outside of the cells where the reaction with chloride can induce damage to adjacent tissue.

Linkage of myeloperoxidase activity to disease has been implicated in neurological diseases with a neuroinflammatory response including multiple sclerosis, Alzheimer's disease, Parkinson's disease and stroke as well as other inflammatory diseases or conditions like asthma, chronic obstructive pulmonary disease, cystic fibrosis, atherosclerosis, ischemic heart disease, heart failure, inflammatory bowel disease, renal glomerular damage and rheumatoid arthritis. Lung cancer has also been suggested to be associated with high MPO levels.

Multiple Sclerosis (MS)

MPO positive cells are immensely present in the circulation and in tissue undergoing inflammation. More specifically MPO containing macrophages and microglia has been documented in the CNS during disease; multiple sclerosis, Parkinson's disease and Alzheimer's disease. It is supposed that some aspects of a chronic ongoing inflammation result in an overwhelming destruction where agents from MPO reactions have an important role.

The enzyme is released both extracellularly as well as into phagolysosomes in the neutrophils. A prerequisite for the MPO activity is the presence of hydrogen peroxide, generated by NADPH oxidase and a subsequent superoxide dismutation. The oxidized enzyme is capable to use a plethora of different substrates of which chloride is most recognized. From this reaction the strong non-radical oxidant—hypochlorous acid (HOCl)—is formed. HOCl oxidizes sulphur containing amino acids like cysteine and methionine very efficiently. It also forms chloramines with amino groups, both in proteins and other biomolecules. It chlorinates phenols (like tyrosine) and unsaturated bonds in lipids, oxidizes iron centers and crosslinks proteins.

Proteolytic cascades participate both in cell infiltration through the BBB as well as the destruction of BBB, myelin and nerve cells. Activation of matrix metalloproteinases (MMPs) can be accomplished through the action of upstream proteases in a cascade as well as through oxidation of a disulfide bridge. This oxidation can be either a nitrosylation or HOCl-mediated oxidation. Both reactions can be a consequence of MPO activity. Several reports have suggested a role for MMP's in general and MMP-9 in particular as influencing cell infiltration as well as tissue damage (BBB breakdown and demyelination), both in MS and EAE. The importance of these specific kinds of mechanisms in MS comes from studies where increased activity and presence of proteases have been identified in MS brain tissue and CSF. Supportive data has also been generated by doing EAE studies with mice deficient in some of the proteases implicated to participate in the MS pathology, or by using pharmacological approaches.

The demyelination is supposed to be dependent on the cytotoxic T-cells and toxic products generated by activated phagocytes. The axonal loss is thus influenced by proteases and reactive oxygen and nitrogen intermediates. When MPO is present it will obviously have the capability of both activating proteases (directly as well as through disinhibition by influencing protease inhibitors) and generating reactive species.

Chronic Obstructive Pulmonary Disease (COPD)

Chronic obstructive pulmonary disease (COPD) is a disease state characterised by airflow limitation that is not fully reversible. The airflow limitation is usually both progressive and associated with an abnormal inflammatory response of the lungs to noxious particles or gases. COPD is a major public health problem. It is the fourth leading cause of chronic morbidity and mortality in the United States and is projected to rank fifth in 2020 as a worldwide burden of disease. In the UK the prevalence of COPD is 1.7% in men and 1.4% in women. COPD spans a range of severity from mild to very severe, with the cost of treatment rising rapidly as the severity increases.

Levels of MPO in sputum and BAL are much greater in COPD patients than normal, non-smoking controls. MPO levels are further elevated during exacerbations of the disease. The role of MPO is likely to be more important in exacerbations of COPD.

In addition to the destructive capacity of MPO there is a strong clinical link with vascular disease. Dysfunctional MPO polymorphisms are associated with a reduced risk of mortality from coronary artery disease, and patients with high serum levels of MPO have increased risk of acute coronary syndrome. The effects of MPO on vascular disease may extend to COPD, since there is strong evidence that the pulmonary vasculature is one of the earliest sites of involvement in the smokers' lung. Striking changes in the intima of the pulmonary arteries have been described which show a dose relationship with smoking. The physiological function of MPO is associated with innate host defence. This role, however, is not critical as most cases of MPO deficient patients have relatively benign symptoms. In summary, there is considerable evidence that elevated MPO levels in COPD may contribute to the disease via several mechanisms. A selective inhibitor of MPO would therefore be expected to alleviate both the acute and chronic inflammatory aspects of COPD and may reduce the development of emphysema.

Atherosclerosis

An MPO inhibitor should reduce the atherosclerotic burden and/or the vulnerability of existing atherosclerotic lesions and thereby decrease the risk of acute myocardial infarction, unstable angina or stroke, and reduce ischemia/reperfusion injury during acute coronary syndrome and ischemic cerebrovascular events. Several lines of data support a role for MPO in atherosclerosis. MPO is expressed in the shoulder regions and necrotic core of human atherosclerotic lesions and active enzyme has been isolated from autopsy specimens of human lesions. In eroded and ruptured human lesions, as compared to fatty streaks, an increased number of MPO expressing macrophages have been demonstrated, suggesting a particular role for MPO in acute coronary syndromes. Patients with established coronary artery disease have higher plasma and leukocyte MPO levels than healthy controls. Moreover, in two large prospective studies plasma levels of MPO predicted the risk of future coronary events or revascularisation. Total MPO deficiency in humans has a prevalece of 1 in 2000-4000 individuals. These individuals appear principally healthy but a few cases of severe Candida infection have been reported. Interestingly, MPO deficient humans are less affected by cardiovascular disease than controls with normal MPO levels. A polymorphism in the MPO promoter affects expression leading to high and low MPO expressing individuals. In three different studies the high expression genotype has been associated with an increased risk of cardiovascular disease. Data accumulated during the last ten years indicate that the proatherogenic actions of MPO include oxidation of lipoproteins, induction of endothelial dysfunction via consuming nitric oxide and destabilisation of atherosclerotic lesions by activation of proteases. Recently, several studies have focused on nitro- and chlorotyrosine modifications of LDL and HDL lipoproteins. Since chlorotyrosine modifications in vivo only can be generated by hypochlorus acid produced by MPO these modifications are regarded as specific markers of MPO activity. LDL particles exposed to MPO in vitro become aggregated, leading to facilitated uptake via macrophage scavenger receptors and foam cell formation. Chlorotyrosine modification of apoA1, the main apolipoprotein of HDL cholesterol, results in impaired cholesterol acceptor function. Systematic studies of these mechanisms have shown that MPO binds to and travels with apoA1 in plasma. Moreover, MPO specifically targets those tyrosine residues of apoA1 that physically interact with the macrophage ABCA1 cassette transporter during cholesterol efflux from the macrophage. Thus, MPO seems to have a dual aggravating role in atherosclerotic lesions, i.e. increasing lipid accumulation via aggregation of LDL particles and decreasing the reverse cholesterol transport via attack on the HDL protein apoA1.

The present invention discloses novel thioxanthines that display useful properties as inhibitors of the enzyme MPO. Furthermore, the novel compounds of the present invention display either one or more than one of the following: (i) improved selectivity towards TPO; (ii) unexpectedly high inhibitory activity towards MPO; and (iii) improved brain permeability; (iv) improved solubility and/or (v) improved half-life when compared to known thioxanthines, such as, for example, thioxanthines disclosed in WO 03/089430 and WO 05/037835.

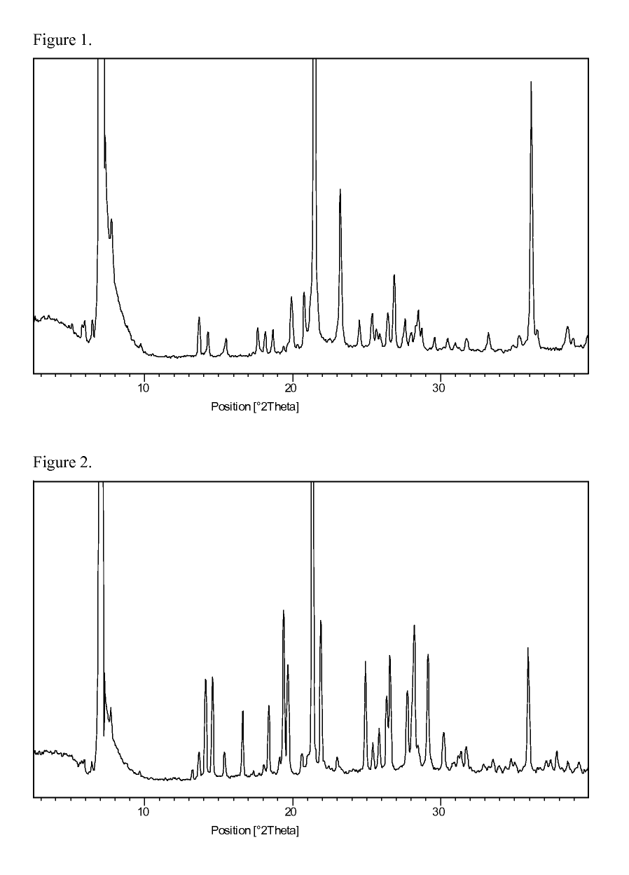

FIG. 1 shows the XRPD pattern of 3-(2R-Tetrahydrofuryl-methyl)-2-thioxanthine Form A.

FIG. 2 shows the XRPD pattern of 3-(2R-Tetrahydrofuryl-methyl)-2-thioxanthine Form B.

It has been found that 3-(2R-tetrahydrofuryl-methyl)-2-thioxanthine can exist in more than one crystal form. The compounds are hereinafter referred to as 3-(2R-tetrahydrofuryl-methyl)-2-thioxanthine Forms A to B. The notation A to B relates to the order in time in which the forms were created, not to their relative thermodynamic stability.

As used herein, the term “substantially pure” means the crystalline form of 3-(2R-tetrahydrofuryl-methyl)-2-thioxanthine referred to contains at least about 90 wt. %, based on the weight of such 3-(2R-tetrahydrofuryl-methyl)-2-thioxanthine crystalline form. For example, if the term substantially pure is used in connection with a crystalline form of 3-(2R-tetrahydrofuryl-methyl)-2-thioxanthine comprising 3-(2R-tetrahydrofuryl-methyl)-2-thioxanthine Form A, the crystalline form of 3-(2R-tetrahydrofuryl-methyl)-2-thioxanthine contains at least about 90 wt. % 3-(2R-tetrahydrofuryl-methyl)-2-thioxanthine Form A, based on the weight of the crystalline form of 3-(2R-tetrahydrofuryl-methyl)-2-thioxanthine. The term “at least about 90 wt. %,” while not intending to limit the applicability of the doctrine of equivalents to the scope of the claims, includes, but is not limited to, for example, about 90, 90, about 91, 91, about 92, 92, about 93, 93, about 94, 94, about 95, 95, about 96, 96, about 97, 97, about 98, 98, about 99, 99, and about 100 wt. %, based on the weight of the crystalline form referred to. The remainder of the crystalline form of Formula (I) may comprise other Form(s) of Formula (I) and/or reaction impurities and/or processing impurities that arise, for example, when the crystalline form is prepared.

A crystalline form of 3-(2R-tetrahydrofuryl-methyl)-2-thioxanthine may be deemed substantially pure if the crystalline form contains at least 90 wt. %, based on the weight of such of 3-(2R-tetrahydrofuryl-methyl)-2-thioxanthine crystalline form as measured by means that are at this time known and generally accepted in the art, of a crystalline form selected from 3-(2R-tetrahydrofuryl-methyl)-2-thioxanthine Form A and 3-(2R-tetrahydrofuryl-methyl)-2-thioxanthine Form B; and less than about 10 wt. %, based on the weight of such 3-(2R-tetrahydrofuryl-methyl)-2-thioxanthine crystalline form, of material comprising other crystalline form(s) of 3-(2-tetrahydrofuryl-methyl)-2-thioxanthine and/or reaction impurities and/or processing impurities.

The presence of reaction impurities and/or processing impurities may be determined by analytical techniques known in the art, such as, for example, chromatography, nuclear magnetic resonance spectroscopy, mass spectrometry, and/or infrared spectroscopy.

All numbers expressing quantities of ingredients, weight percentages, temperatures, and so forth that are preceded by the word “about” are to be understood as only approximations so that slight variations above and below the stated number may be used to achieve substantially the same results as the stated number. Accordingly, unless indicated to the contrary, numerical parameters preceded by the word “about” are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

One aspect of the present invention provides 3-(2R-tetrahydrofuryl-methyl)-2-thioxanthine Form A.

3-(2R-tetrahydrofuryl-methyl)-2-thioxanthine Form A, according to the present invention, is characterized by an X-ray powder diffraction (XRPD) pattern, as in FIG. 1, exhibiting substantially the angles, d-values and intensities set forth in Table 1:

TABLE 1

| Angle | | Relative |
|---|---|---|
| (°2theta) | d-value (Å) | intensity |
| 7.11 | 12.42 | vs |
| 13.61 | 6.50 | m |
| 14.24 | 6.22 | w |
| 15.43 | 5.74 | w |
| 17.58 | 5.04 | m |
| 18.09 | 4.90 | m |
| 18.61 | 4.76 | m |
| 19.87 | 4.47 | m |
| 20.71 | 4.29 | m |
| 21.42 | 4.15 | s |
| 23.16 | 3.84 | m |
| 24.46 | 3.64 | m |
| 25.29 | 3.52 | m |
| 26.37 | 3.38 | m |
| 26.80 | 3.32 | m |
| 27.53 | 3.24 | m |
| 27.92 | 3.19 | w |
| 28.26 | 3.16 | m |
| 28.43 | 3.14 | m |
| 29.53 | 3.02 | w |
| 30.41 | 2.94 | w |
| 30.92 | 2.89 | w |
| 31.67 | 2.82 | w |
| 33.16 | 2.70 | w |
| 36.06 | 2.49 | m |
| 36.45 | 2.46 | w |
| 38.50 | 2.34 | w |

The peaks, identified with d-values calculated from the Bragg formula and intensities, have been extracted from the diffractogram of 3-(2R-tetrahydrofuryl-methyl)-2-thioxanthine Form A. The relative intensities are less reliable and instead of numerical values the following definitions are used:

| % Relative Intensity* | Definition |
|---|---|
| 25-100 | vs (very strong) |
| 4-25 | s (strong) |
| 0.5-4 | m (medium) |
| 0-0.4 | w (weak) |

*The relative intensities are derived from diffractograms measured with variable slits.

3-(2R-tetrahydrofuryl-methyl)-2-thioxanthine Form A is a crystalline form exhibiting advantageous properties, such as convenient handling as well as chemical and solid-state stability.

Another aspect of the present invention provides 3-(2R-tetrahydrofuryl-methyl)-2-thioxanthine Form B.

3-(2R-tetrahydrofuryl-methyl)-2-thioxanthine Form B, according to the present invention, is characterized in providing an XRPD pattern, as in FIG. 2, exhibiting substantially the angles, d-values and intensities set forth in Table 2:

TABLE 2

| Angle | | Relative |
|---|---|---|
| (°2theta) | d-value (Å) | intensity |
| 7.08 | 12.47 | vs |
| 13.62 | 6.50 | w |
| 14.06 | 6.29 | m |
| 14.54 | 6.09 | m |
| 15.35 | 5.77 | w |
| 16.56 | 5.35 | m |
| 18.32 | 4.84 | m |
| 19.05 | 4.66 | w |
| 19.34 | 4.59 | m |
| 19.64 | 4.52 | m |
| 20.57 | 4.31 | w |
| 21.30 | 4.17 | s |
| 21.85 | 4.06 | m |
| 22.97 | 3.87 | w |
| 24.86 | 3.58 | m |
| 25.36 | 3.51 | w |
| 25.78 | 3.45 | m |
| 26.30 | 3.39 | m |
| 26.52 | 3.36 | m |
| 27.70 | 3.22 | m |
| 28.02 | 3.18 | m |
| 28.14 | 3.17 | m |
| 29.07 | 3.07 | m |
| 30.13 | 2.96 | w |
| 31.31 | 2.85 | w |
| 31.68 | 2.82 | w |
| 33.47 | 2.68 | w |
| 35.86 | 2.50 | m |

The peaks, identified with d-values calculated from the Bragg formula and intensities, have been extracted from the diffractogram of 3-(2R-tetrahydrofuryl-methyl)-2-thioxanthine Form A. The relative intensities are less reliable and instead of numerical values the following definitions are used:

| % Relative intensity* | Definition |
|---|---|
| 25-100 | vs (very strong) |
| 5-25 | s (strong) |
| 0.6-5 | m (medium) |
| 0-0.5 | w (weak) |

*The relative intensities are derived from diffractograms measured with variable slits.

3-(2R-tetrahydrofuryl-methyl)-2-thioxanthine Form B is a crystalline form exhibiting advantageous properties, such as convenient handling as well as chemical and solid-state stability.

3-(2R-tetrahydrofuryl-methyl)-2-thioxanthine Form A is more stable at ambient temperature, such as room temperature, while 3-(2R-tetrahydrofuryl-methyl)-2-thioxanthine Form B is more stable at temperatures over +65° C.

It is possible to crystallize 3-(2R-tetrahydrofuryl-methyl)-2-thioxanthine, i.e. the compounds of the present invention in one single solvent or in a mixture of solvents. An example of a suitable solvent is DMSO.

Crystallization of the compounds of the present invention from an appropriate solvent system, containing at least one solvent, may be achieved by attaining supersaturation in a solvent system by solvent evaporation, by temperature decrease, and/or via the addition of anti-solvent (i.e. a solvent in which the compounds of the invention are poorly soluble). An example of a suitable antisolvent is a mixture of water and ethanol.

Crystallization may also be initiated and/or effected with or without seeding with crystals of the appropriate crystalline compound of the invention.

Crystallization of compounds of the present invention can be achieved starting from pure 3-(2R-tetrahydrofuryl-methyl)-2-thioxanthine of any form, or mixtures of any form.

Whether anhydrate or solvate crystallizes is related to the kinetics and equilibrium conditions of the respective forms at the specific conditions. Thus, the crystalline form that is obtained depends upon both the kinetics and the thermodynamics of the crystallization process. Under certain thermodynamic conditions (solvent system, temperature, pressure and concentration of compound of the invention), one crystalline form may be more stable than another (or indeed any other). However, crystalline forms that have a relatively low thermodynamic stability may be kinetically favored. Thus, in addition, kinetic factors, such as time, impurity profile, agitation, the presence or absence of seeds, etc. may also influence which form crystallizes.

Another aspect of the present invention provides processes for the preparation of 3-(2R-tetrahydrofuryl-methyl)-2-thioxanthine Forms A to B.

3-(2R-tetrahydrofuryl-methyl)-2-thioxanthine Form A is obtained upon crystallization from 3-(2R-tetrahydrofuryl-methyl)-2-thioxanthine crude in temperatures about +60° C. or below, for example at room temperature by, for example, addition of an antisolvent. The obtained form is stable.

3-(2R-tetrahydrofuryl-methyl)-2-thioxanthine Form B is obtained upon crystallization from 3-(2R-tetrahydrofuryl-methyl)-2-thioxanthine at temperatures about +60° C. or above by, for example, addition of an antisolvent.

The compounds of the invention may be administered and used as described in WO03/089430.

The compounds of the invention may be further processed before formulation into a suitable pharmaceutical formulation. For example, the crystalline form may be milled or ground into smaller particles.

Another aspect of the present invention provides a pharmaceutical formulation including a compound of the invention in admixture with at least one pharmaceutically acceptable adjuvant, diluent or carrier.

Another aspect of the present invention provides a pharmaceutical formulation comprising a mixture of 3-(2R-tetrahydrofuryl-methyl)-2-thioxanthine Form A and 3-(2R-tetrahydrofuryl-methyl)-2-thioxanthine Form B in admixture with at least one pharmaceutically acceptable excipient.

Another aspect of the present invention provides a method of treatment of a condition where 3-(2R-tetrahydrofuryl-methyl)-2-thioxanthine is required or desired, which method includes administering a therapeutically effective amount of a compound of the invention to a patient in need of such treatment.

Another aspect of the present invention provides the use of 3-(2R-tetrahydrofuryl-methyl)-2-thioxanthine Form A or a pharmaceutically acceptable salt thereof, in the manufacture of a medicament, for the treatment or prophylaxis of diseases or conditions in which inhibition of the enzyme MPO is beneficial.

Another aspect of the present invention provides the use of 3-(2R-tetrahydrofuryl-methyl)-2-thioxanthine Form A, or a pharmaceutically acceptable salt thereof, in the manufacture of a medicament, for the treatment or prophylaxis of neuroinflammatory disorders, cardio- and cerebrovascular atherosclerotic disorders and peripheral artery disease, heart failure and respiratory disorders such as chronic obstructive pulmonary disease (COPD); bronchitis, including infectious and eosinophilic bronchitis; emphysema; bronchiectasis or cystic fibrosis.

Another aspect of the present invention provides the use of 3-(2R-tetrahydrofuryl-methyl)-2-thioxanthine Form A, or a pharmaceutically acceptable salt thereof, in the manufacture of a medicament, for the treatment or prophylaxis of multiple sclerosis. Treatment may include slowing progression of disease.

Another aspect of the present invention provides the use of 3-(2R-tetrahydrofuryl-methyl)-2-thioxanthine Form A, or a pharmaceutically acceptable salt thereof, in the manufacture of a medicament, for the treatment or prophylaxis of Parkinson's disease. Treatment may include slowing progression of disease.

Another aspect of the present invention provides the use of 3-(2R-tetrahydrofuryl-methyl)-2-thioxanthine Form A or a pharmaceutically acceptable salt thereof, in the manufacture of a medicament, for the treatment or prophylaxis of atherosclerosis by preventing and/or reducing the formation of new atherosclerotic lesions or plaques and/or by preventing or slowing progression of existing lesions and plaques.

Another aspect of the present invention provides use of 3-(2R-tetrahydrofuryl-methyl)-2-thioxanthine Form A or a pharmaceutically acceptable salt thereof, in the manufacture of a medicament, for the treatment or prophylaxis of atherosclerosis by changing the composition of the plaques to reduce the risk of plaque rupture and atherothrombotic events.

Another aspect of the present invention provides the use of 3-(2R-tetrahydrofuryl-methyl)-2-thioxanthine Form A or a pharmaceutically acceptable salt thereof, in the manufacture of a medicament, for the treatment or prophylaxis of respiratory disorders, such as chronic obstructive pulmonary disease. Treatment may include slowing progression of disease.

Another aspect of the present invention provides a method of treating, or reducing the risk of, diseases or conditions in which inhibition of the enzyme MPO is beneficial which comprises administering to a person suffering from or at risk of, said disease or condition, a therapeutically effective amount of 3-(2R-tetrahydrofuryl-methyl)-2-thioxanthine Form A, or a pharmaceutically acceptable salt thereof.

Another aspect of the present invention provides a method of treating, or reducing the risk of, neuroinflammatory disorders, cardio- and cerebrovascular atherosclerotic disorders or peripheral artery disease, or heart failure or respiratory disorders, such as chronic obstructive pulmonary disease (COPD), in a person suffering from or at risk of, said disease or condition, wherein the method comprises administering to the person a therapeutically effective amount of 3-(2R-tetrahydrofuryl-methyl)-2-thioxanthine Form A, or a pharmaceutically acceptable salt thereof. According to one embodiment of the present invention said COPD is bronchitis, including infectious and eosinophilic bronchitis; emphysema; bronchiectasis or cystic fibrosis.

Another aspect of the present invention provides a method of treating, or reducing the risk of, multiple sclerosis in a person suffering from or at risk of, said disease or condition, wherein the method comprises administering to the person a therapeutically effective amount of 3-(2R-tetrahydrofuryl-methyl)-2-thioxanthine Form A, or a pharmaceutically acceptable salt thereof.

Another aspect of the present invention provides a method of treating, or reducing the risk of, Parkinson's disease in a person suffering from or at risk of, said disease or condition, wherein the method comprises administering to the person a therapeutically effective amount of 3-(2R-tetrahydrofuryl-methyl)-2-thioxanthine Form A, or a pharmaceutically acceptable salt thereof.

Another aspect of the present invention provides a method of treating, or reducing the risk of atherosclerosis by preventing and/or reducing the formation of new atherosclerotic lesions or plaques and/or by preventing or slowing progression of existing lesions and plaques in a person suffering from or at risk of, said disease or condition, wherein the method comprises administering to the person a therapeutically effective amount of 3-(2R-tetrahydrofuryl-methyl)-2-thioxanthine Form A or a pharmaceutically acceptable salt thereof.

Another aspect of the present invention provides a method of treating, or reducing the risk of atherosclerosis by changing the composition of the plaques so as to reduce the risk of plaque rupture and atherothrombotic events in a person suffering from or at risk of, said disease or condition, wherein the method comprises administering to the person a therapeutically effective amount of 3-(2R-tetrahydrofuryl-methyl)-2-thioxanthine Form A or a pharmaceutically acceptable salt thereof

Another aspect of the present invention provides a pharmaceutical formulation comprising a therapeutically effective amount of 3-(2R-tetrahydrofuryl-methyl)-2-thioxanthine Form A, or a pharmaceutically acceptable salt thereof, in admixture with a pharmaceutically acceptable adjuvant, diluent or carrier, for use in the treatment or prophylaxis of diseases or conditions in which inhibition of the enzyme MPO is beneficial.

Another aspect of the present invention provides a pharmaceutical formulation comprising a therapeutically effective amount of 3-(2R-tetrahydrofuryl-methyl)-2-thioxanthine Form A, or a pharmaceutically acceptable salt thereof, in admixture with a pharmaceutically acceptable adjuvant, diluent or carrier, for use in the treatment or prophylaxis of neuroinflammatory disorders.

Another aspect of the present invention provides a pharmaceutical formulation comprising a therapeutically effective amount of 3-(2R-tetrahydrofuryl-methyl)-2-thioxanthine Form A, or a pharmaceutically acceptable salt thereof, in admixture with a pharmaceutically acceptable adjuvant, diluent or carrier, for use in the treatment or prophylaxis of multiple sclerosis, cardio- and cerebrovascular atherosclerotic disorders and peripheral artery disease and heart failure and respiratory disorders, such as chronic obstructive pulmonary disease.

Another aspect of the present invention provides a pharmaceutical formulation comprising a therapeutically effective amount of 3-(2R-tetrahydrofuryl-methyl)-2-thioxanthine Form A, or a pharmaceutically acceptable salt thereof, in admixture with a pharmaceutically acceptable adjuvant, diluent or carrier, for use in the treatment or prophylaxis of atherosclerosis by preventing and reducing the formation of new atherosclerotic lesions and/or plaques and/or by preventing or slowing progression of existing lesions and plaques.

Another aspect of the present invention provides a pharmaceutical formulation comprising a therapeutically effective amount of 3-(2R-tetrahydrofuryl-methyl)-2-thioxanthine Form A, or a pharmaceutically acceptable salt thereof, in admixture with a pharmaceutically acceptable adjuvant, diluent or carrier, for use in the treatment or prophylaxis of atherosclerosis by changing the composition of the plaques so as to reduce the risk of plaque rupture and atherothrombotic events.

Another aspect of the present invention provides use of a mixture of 3-(2R-tetrahydrofuryl-methyl)-2-thioxanthine Form A and 3-(2R-tetrahydrofuryl-methyl)-2-thioxanthine Form B as active ingredients in the manufacture of a medicament for use in treatment or prophylaxis of diseases or conditions in which inhibition of the enzyme MPO is beneficial. Another aspect of the present invention provides a method of treatment or prophylaxis of diseases or conditions in which inhibition of the enzyme MPO is beneficial, which comprises administration of a therapeutically effective amount of a mixture of 3-(2R-tetrahydrofuryl-methyl)-2-thioxanthine Form A and 3-(2R-tetrahydrofuryl-methyl)-2-thioxanthine Form B, to a patient suffering therefrom.

The present invention further relates to therapies for the treatment of: Neurodegenerative Disorder(s) including but not limited to Alzheimer's Disease (AD), Dementia, Cognitive Deficit in Schizophrenia (CDS), Mild Cognitive Impairment (MCI), Age-Associated Memory Impairment (AAMI), Age-Related Cognitive Decline (ARCD), Cognitive Impairement No Dementia (CIND), Multiple Sclerosis, Parkinson's Disease (PD), postencephalitic parkinsonism, Huntington's Disease, amyotrophic lateral sclerosis (ALS), motor neuron diseases (MND), Multiple System Atrophy (MSA), Corticobasal Degeneration, Progressive Supranuclear Paresis, Guillain-Barr\acute{3} Syndrome (GBS), and Chronic Inflammatory Demyelinating Polyneuropathy (CIDP). Dementia includes, but is not limited to, Down syndrome, vascular dementia, dementia with Lewy bodies, HIV dementia, Frontotemporal dementia Parkinson's Type (FTDP), Pick's Disease, Niemann-Pick's Disease, traumatic brain injury (TBI), dementia pugilistica, Creutzfeld-Jacob Disease and prion diseases.

The present invention further relates to therapies for the treatment of: Neuroinflammatory Disorder(s)including but not limited to Multiple Sclerosis (MS), Parkinson's disease, Multiple System Atrophy (MSA), Corticobasal Degeneration, Progressive Supranuclear Paresis, Guillain-Barré Syndrome (GBS), chronic inflammatory demyelinating polyneuropathy (CIDP). Multiple sclerosis (MS) includes Relapse Remitting Multiple Sclerosis (RRMS), Secondary Progressive Multiple Sclerosis (SPMS), and Primary Progressive Multiple Sclerosis (PPMS).

The present invention further relates to therapies for the treatment of: Cognitive Disorder(s) including but not limited to

a) Dementia, including but not limited to Alzheimer's Disease (AD), Down syndrome, vascular dementia, Parkinson's Disease (PD), postencephelatic parkinsonism, dementia with Lewy bodies, HIV dementia, Huntington's Disease, amyotrophic lateral sclerosis (ALS), motor neuron diseases (MND), Frontotemporal dementia Parkinson's Type (FTDP), progressive supranuclear palsy (PSP), Pick's Disease, Niemann-Pick's Disease, corticobasal degeneration, traumatic brain injury (TBI), dementia pugilistica, Creutzfeld-Jacob Disease and prion diseases;

b) Cognitive Deficit in Schizophrenia (CDS);

c) Mild Cognitive Impairment (MCI);

d) Age-Associated Memory Impairment (AAMI);

e) Age-Related Cognitive Decline (ARCD);

f) Cognitive Impairement No Dementia (CIND).

The present invention further relates to therapies for the treatment of: Attention-Deficit and Disruptive Behavior Disorder(s) including but not limited to attention deficit disorder (ADD), attention deficit hyperactivity disorder (ADHD) and affective disorders.

The present invention also relates to the treatment of the diseases and conditions below which may be treated with the compounds of the present invention: respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus; bone and joints: arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;

The invention further relates to combination therapies wherein 3-(2R-tetrahydrofuryl-methyl)-2-thioxanthine Form A or a pharmaceutically acceptable salt thereof, or a pharmaceutical composition or formulation comprising 3-(2R-tetrahydrofuryl-methyl)-2-thioxanthine Form A is administered concurrently or sequentially with therapy and/or an agent for the treatment of any one of cardio- and cerebrovascular atherosclerotic disorders and peripheral artery disease.

3-(2R-tetrahydrofuryl-methyl)-2-thioxanthine Form A or a pharmaceutically acceptable salt thereof may be administered in association with compounds from one or more of the following groups:

1) anti-inflammatory agents, for example
a) NSAIDs (e.g. acetylsalicylic acid, Ibuprofen, naproxen, flurbiprofen, diclofenac, indometacin);
b) leukotriene synthesis inhibitors (5-LO inhibitors e.g. AZD4407,Zileuton, licofelone, CJ13610, CJ13454; FLAP inhibitors e.g. BAY-Y-1015, DG-031, MK591, MK886, A81834; LTA4 hydrolase inhibitors e.g. SC56938, SC57461A);
c) leukotriene receptor antagonists ( e.g. CP195543, amelubant, LY293111, accolate, MK571);

2) anti-hypertensive agents, for example
a) beta-blockers (e.g. metoprolol, atenolol, sotalol);
b) angiotensin converting enzyme inhibitors (e.g. captopril, ramipril, quinapril, enalapril);
c) calcium channel blockers (e.g. verapamil, diltiazem, felodipine, amlodipine);
d) angiotensin II receptor antagonists (e.g. irbesartan, candesartan, telemisartan, losartan);

3) anti-coagulantia, for example
a) thrombin inhibitors (e.g. ximelagatran), heparines, factor Xa inhibitors;
b) platelet aggregation inhibitors (e.g. clopidrogrel, ticlopidine, prasugel, AZ4160);

4) modulators of lipid metabolism, for example
a) insulin sensitizers such as PPAR agonists (e.g. pioglitazone, rosiglitazone, Galida, muraglitazaar, gefemrozil, fenofibrate);
b) HMG-CoA reductase inhibitors, statins (e.g. simvastatin, pravastatin, atorvaststin, rosuvastatin, fluvastatin);
c) cholesterol absorption inhibitors (e.g. ezetimibe);
d) IBAT inhibitors (e.g. AZD-7806);
e) LXR agonists (e.g. GW-683965A, T-0901317);
f) FXR receptor modulators;
g) phospholipase inhibitors;

5) anti-anginal agents, for example, nitrates and nitrites;

6) modulators of oxidative stress, for example, anti-oxidants (e.g. probucol, AG1067).

3-(2R-tetrahydrofuryl-methyl)-2-thioxanthine Form B could also be used for the above-mentioned aspects of the present invention.

For the avoidance of doubt, “treatment” includes the therapeutic treatment, as well as the prophylaxis, of a condition.

3-(2R-tetrahydrofuryl-methyl)-2-thioxanthine Form A and Form B represent the (−)-enantiomer of 3-(2-tetrahydrofuryl-methyl)-2-thioxanthine.

The compounds of the invention have the advantage that they are in a form that provides for improved ease of handling. Further, the compounds of the invention have the advantage that they may be produced in forms that have improved chemical and solid state stability as well as lower hygroscopicity. Thus, the compounds may be stable when stored over prolonged periods.

The invention is illustrated, but in no way limited, by the following examples.

It will be appreciated by the skilled person that crystalline forms of compounds of the invention may be prepared by analogy with processes described herein and/or in accordance with the Examples below, and may show essentially the same XRPD patterns as those disclosed herein. By “essentially the same” XRPD patterns we include those instances when it is clear from the relevant patterns (allowing for experimental error) that essentially the same crystalline form has been formed. When provided, XRPD 2-θ angle values may vary in the range ±0.05 °2θ. It will be appreciated by the skilled person that XRPD intensities may vary when measured for essentially the same crystalline form for a variety of reasons including, for example, preferred orientation.

Method of Synthesis

3-(2R-tetrahydrofuryl-methyl)-2-thioxanthine crystals may be formed from a solvent solution by addition of an antisolvent or by decreasing pH from a basic solution. Dimethyl sulfoxide (DMSO) is an example of a good solvent, while alcohols and/or water may be used as antisolvent. Alcohols such as ethanol are suitable, in combination with water at a basic pH. In this case, an acid may be used to decrease pH and to precipitate the substance, preferentially Hydrochloric acid. The total amount of solvent may vary between 1 (v/w) to 100 (v/w) volume parts per weight of starting material, preferably between 5 (v/w) to 50 (v/w). The temperature of the reaction/crystallization may be between 0 and 100° C. Two polymorphs have been discovered. Polymorph A is preferably formed at and below 60° C. and polymorph B at and above 60° C.

Stirring substance of polymorph A in a solution about or above 60° C. transforms it to polymorph B, while stirring substance of polymorph B in solutions about or below 60° C. transforms it to polymorph A. Performing the crystallization at or close to 60° C. or starting crystallization above/below 60° C. and then decreasing/increasing temperature below/above 60° C. may result in a mixture of the two polymorphs.

The following examples will describe, but not limit, the invention.

Form A—Alternative 1

3-(2R-tetrahydrofuryl-methyl)-2-thioxanthine crude (Form A+B) (10.0 gram) was dissolved in a solution of ethanol (240 mL), water (100 mL) and sodium hydroxide solution (1 M, 80 mL) at a pH above 12 and at about room temperature. The solution was screen filtered to a small glass reactor.

3-(2R-tetrahydrofuryl-methyl)-2-thioxanthine (Form A) was crystallized by a slow addition of 3 M HCl (26 mL) at room temperature and at good stirring conditions. When crystals appeared the addition was stopped for five minutes. Finally, when all HCl had been added the slurry was stirred at room temperature overnight. Then the slurry was filtered and washed with ethanol and water and dried at vacuum at 40° C. The yield became 9.02 g, 90% and purity of at least 99%. Identity by ₁H NMR (500 MHz, DMSO-d6) δ (ppm) 13.82 (br s, 1H), 12.45(s, 1H), 8.15(s, 1H), 4.58(m, 1H), 4.55/4.41(m, 2H), 3.81/3.60(m, 2H), 1.94/1.80(m, 2H), 1.88/1.74(m, 2H)

Form A—Alternative 2

3-(2R-tetrahydrofuryl-methyl)-2-thioxanthine crude (4.0 gram) was dissolved in a solution of Dimethyl sulfoxide (21 mL), at 65° C.

Crystallization was initialized at 65° C. by addition of antisolvent, a mixture of ethanol and water. At the point where crystals were observed in the solution the addition was stopped. Then the solution was cooled for 2 hrs. to room temperature and then the addition of antisolvent was taken up again at a slow rate. The slurry was filtered and washed. Washing was performed in 6 steps with DMSO/Ethanol/water, with ethanol/water and with ethanol. Then it was dried at vacuum at 40° C. The yield became 3.3 g, 83% and purity of at least 98%. 3-(2R-tetrahydrofuryl-methyl)-2-thioxanthine was achieved in crystal Form A. Identity by ¹H NMR (500 MHz, DMSO-d6) δ (ppm) 13.82 (br s, 1H), 12.45(s, 1H), 8.15(s, 1H), 4.58(m, 1H), 4.55/4.41(m, 2H), 3.81/3.60(m, 2H), 1.94/1.80(m, 2H), 1.88/1.74(m, 2H).

Form A was characterized via an XRPD pattern using CuKα-radiation (1.5406 Å) to obtain substantially the angles, d-values and intensities set forth in Table 3:

TABLE 3

| Angle (° 2theta) | d-value (Å) | Relative intensity |
|---|---|---|
| 7.11 | 12.42 | vs |
| 13.61 | 6.50 | m |
| 14.24 | 6.22 | w |
| 15.43 | 5.74 | w |
| 17.58 | 5.04 | m |
| 18.09 | 4.90 | m |
| 18.61 | 4.76 | m |
| 19.87 | 4.47 | m |
| 20.71 | 4.29 | m |
| 21.42 | 4.15 | s |
| 23.16 | 3.84 | m |
| 24.46 | 3.64 | m |
| 25.29 | 3.52 | m |
| 26.37 | 3.38 | m |
| 26.80 | 3.32 | m |
| 27.53 | 3.24 | m |
| 27.92 | 3.19 | w |
| 28.26 | 3.16 | m |
| 28.43 | 3.14 | m |
| 29.53 | 3.02 | w |
| 30.41 | 2.94 | w |
| 30.92 | 2.89 | w |
| 31.67 | 2.82 | w |
| 33.16 | 2.70 | w |
| 36.06 | 2.49 | m |
| 36.45 | 2.46 | w |
| 38.50 | 2.34 | w |

The peaks, identified with d-values calculated from the Bragg formula and intensities, have been extracted from the diffractogram of 3-(2R-tetrahydrofuryl-methyl)-2-thioxanthine Form A. The relative intensities are less reliable and instead of numerical values the following definitions are used:

| % Relative intensity* | Definition |
|---|---|
| 25-100 | Vs (very strong) |
| 4-25 | s (strong) |
| 0.5-4 | M (mediium) |
| 0-0.4 | W (weak) |

*The relative intensities are derived from diffractograms measured with variable slits.

Form B

0.5 gram of 3-(2R-tetrahydrofuryl-methyl)-2-thioxanthine, containing a mixture of Form A and B, was slurried at 90° C. in a mixture of 6 mL ethanol and 2 mL water. After 10 days the transformation to Form B was complete. The sample (about 0.2 gr.) was filtered, washed with ethanol and air-dried. Identity by ¹H NMR (500 MHz, DMSO-d6) δ (ppm) 13.82(br s, 1H), 12.45(s, 1H), 8.15(s, 1H), 4.58(m, 1H), 4.55/4.41(m, 2H), 3.81/3.60(m, 2H), 1.94/1.80(m, 2H), 1.88/1.74(m, 2H).

Form B was characterized via an XRPD using CuKα-radiation (1.5406 Å) to obtain substantially the angles, d-values and intensities set forth in Table 4:

TABLE 4

| Angle (°2theta) | d-value (Å) | Relative intensity |
|---|---|---|
| 7.08 | 12.47 | vs |
| 13.62 | 6.50 | w |
| 14.06 | 6.29 | m |
| 14.54 | 6.09 | m |
| 15.35 | 5.77 | w |
| 16.56 | 5.35 | m |
| 18.32 | 4.84 | m |
| 19.05 | 4.66 | w |
| 19.34 | 4.59 | m |
| 19.64 | 4.52 | m |
| 20.57 | 4.31 | w |
| 21.30 | 4.17 | s |
| 21.85 | 4.06 | m |
| 22.97 | 3.87 | w |
| 24.86 | 3.58 | m |
| 25.36 | 3.51 | w |
| 25.78 | 3.45 | m |
| 26.30 | 3.39 | m |
| 26.52 | 3.36 | m |
| 27.70 | 3.22 | m |
| 28.02 | 3.18 | m |
| 28.14 | 3.17 | m |
| 29.07 | 3.07 | m |
| 30.13 | 2.96 | w |
| 31.31 | 2.85 | w |
| 31.68 | 2.82 | w |
| 33.47 | 2.68 | w |
| 35.86 | 2.50 | m |

The peaks, identified with d-values calculated from the Bragg formula and intensities, have been extracted from the diffractogram of 3-(2R-tetrahydrofuryl-methyl)-2-thioxanthine Form B. The relative intensities are less reliable and instead of numerical values the following definitions are used:

| % Relative intensity* | Definition |
|---|---|
| 25-100 | vs (very strong) |
| 5-25 | s (strong) |
| 0.6-5 | m (mediium) |
| 0-0.5 | w (weak) |

*The relative intensities are derived from diffractograms measured with variable slits.

1. 3-(2R-tetrahydrofuryl-methyl)-2-thioxanthine Form A.

2. 3-(2R-tetrahydrofuryl-methyl)-2-thioxanthine Form A, characterized by an X-ray powder diffraction pattern exhibiting substantially the following d-values:
Form A

| d-value (Å) | Relative intensity |
|---|---|
| 12.42 | vs |
| 6.50 | m |
| 6.22 | w |
| 5.74 | w |
| 5.04 | m |
| 4.90 | m |
| 4.76 | m |
| 4.47 | m |
| 4.29 | m |
| 4.15 | s |
| 3.84 | m |
| 3.64 | m |
| 3.52 | m |
| 3.38 | m |
| 3.32 | m |
| 3.24 | m |
| 3.19 | w |
| 3.16 | m |
| 3.14 | m |
| 3.02 | w |
| 2.94 | w |
| 2.89 | w |
| 2.82 | w |
| 2.70 | w |
| 2.49 | m |
| 2.46 | w |
| 2.34 | w |

3. 3-(2R-tetrahydrofuryl-methyl)-2-thioxanthine Form A, characterized by an X-ray powder diffraction pattern comprising at least one °2θ-value selected from 23.16.

4. 3-(2R-tetrahydrofuryl-methyl)-2-thioxanthine Form A according to claim 3, characterized by an X-ray powder diffraction pattern comprising 3 or more °2θ-values selected from 23.16, 24.46 and 26.80.

5. 3-(2R-tetrahydrofuryl-methyl)-2-thioxanthine Form A according to claim 3, characterized by an X-ray powder diffraction pattern comprising 5 or more °2θ-values selected from 18.61, 19.87, 23.16, 24.46 and 26.80.

6. A process for preparing 3-(2R-tetrahydrofuryl-methyl)-2-thioxanthine Form A according to claim 1, comprising:
a) dissolving or suspending at least one form of 3-(2R-tetrahydrofuryl-methyl)-2-thioxanthine, or mixtures thereof in a suitable solvent;
b) allowing the solution to crystallize;
c) isolating at least one 3-(2R-tetrahydrofuryl-methyl)-2-thioxanthine crystal obtained.

7. A process according to claim 6, wherein step a) is performed at a temperature of 60° C. or below.

8. A process according to claim 6, wherein step b) is performed at a temperature of 60° C. or below.

9. A process according to claim 6, wherein step a) is performed during a prolonged time period.

10. A process according to claim 6, wherein step b) is performed during a prolonged time period.

11. 3-(2R-tetrahydrofuryl-methyl)-2-thioxanthine Form B.

12. 3-(2R-tetrahydrofuryl-methyl)-2-thioxanthine Form B, characterized by an X-ray powder diffraction pattern exhibiting substantially the following d-values:
Form B

| d-value (Å) | Relative intensity |
|---|---|
| 12.47 | vs |
| 6.50 | w |
| 6.29 | m |
| 6.09 | m |
| 5.77 | w |
| 5.35 | m |
| 4.84 | m |
| 4.66 | w |
| 4.59 | m |
| 4.52 | m |
| 4.31 | w |
| 4.17 | s |
| 4.06 | m |
| 3.87 | w |
| 3.45 | m |
| 3.39 | m |
| 3.36 | m |
| 3.22 | m |
| 3.18 | m |
| 3.17 | m |
| 3.07 | m |
| 2.96 | w |
| 2.85 | w |
| 2.82 | w |
| 2.68 | w |
| 2.50 | m |

13. 3-(2R-tetrahydrofuryl-methyl)-2-thioxanthine Form B, characterized by an X-ray powder diffraction pattern comprising at least one °2θ-value selected from 16.56.

14. 3-(2R-tetrahydrofuryl-methyl)-2-thioxanthine Form B according to claim 13, characterized by an X-ray powder diffraction pattern comprising 3 or more °2θ-values selected from 16.56, 24.86 and 29.07.

15. 3-(2R-tetrahydrofuryl-methyl)-2-thioxanthine Form B according to claim 13, characterized by an X-ray powder diffraction pattern comprising 5 or more °2θ-values selected from 14.54, 16.56, 21.85, 24.86 and 29.07.

16. A process for preparing 3-(2R-tetrahydrofuryl-methyl)-2-thioxanthine Form B according to claim 11 comprising:
a) dissolving or suspending at least one form of 3-(2R-tetrahydrofuryl-methyl)-2-thioxanthine, or a mixture thereof in a suitable solvent;
b) allowing the solution to crystallize;
c) isolating at least one 3-(2R-tetrahydrofuryl-methyl)-2-thioxanthine crystal obtained.

17. A process according to claim 16, wherein step a) is performed at a temperature of 60° C. or above.

18. A process according to claim 16, wherein step b) is performed at a temperature of 60° C. or above.

19. A process according to claim 16, wherein step a) is performed during a prolonged time period.

20. A process according to claim 16, wherein step b) is performed during a prolonged time period.

21. 3-(2R-tetrahydrofuryl-methyl)-2-thioxanthine prepared according to claim 6.

22. 3-(2R-tetrahydrofuryl-methyl)-2-thioxanthine prepared according to claim 16.

23. A pharmaceutical formulation comprising at least one crystalline form of 3-(2R-tetrahydrofuryl-methyl)-2-thioxanthine selected from 3-(2R-tetrahydrofuryl-methyl)-2-thioxanthine Form A, 3-(2R-tetrahydrofuryl-methyl)-2-thioxanthine Form B, and mixtures thereof in admixture with at least one pharmaceutically acceptable excipient.

24. The pharmaceutical formulation of claim 23, wherein said formulation consists essentially of 3-(2R-tetrahydrofuryl-methyl)-2-thioxanthine Form A in admixture with at least one pharmaceutically acceptable excipient.

25. The pharmaceutical formulation of claim 23, wherein said formulation consists essentially of 3-(2R-tetrahydrofuryl-methyl)-2-thioxanthine Form B in admixture with at least one pharmaceutically acceptable excipient.

26. The pharmaceutical formulation of claim 23, wherein the at least one crystalline form is 3-(2R-tetrahydrofuryl-methyl)-2-thioxanthine Form A.

27. The pharmaceutical formulation of claim 26, wherein the at least one crystalline form is in a substantially pure form.

28. The pharmaceutical formulation of claim 23, wherein the at least one crystalline form is 3-(2R-tetrahydrofuryl-methyl)-2-thioxanthine Form B.

29. The pharmaceutical formulation of claim 28, wherein the at least one crystalline form is in a substantially pure form.

30. A method of treatment or prophylaxis of at least one disease or condition in which inhibition of the enzyme MPO is beneficial, comprising administering a therapeutically effective amount of at least one crystalline form of 3-(2R-tetrahydrofuryl-methyl)-2-thioxanthine selected from 3-(2R-tetrahydrofuryl-methyl)-2-thioxanthine Form A, 3-(2R-tetrahydrofuryl-methyl)-2-thioxanthine Form B, and mixtures thereof to a patient suffering therefrom.

31. The method of claim 30, wherein the at least one crystalline form of 3-(2R-tetrahydrofuryl-methyl)-2-thioxanthine consists essentially of 3-(2R-tetrahydrofuryl-methyl)-2-thioxanthine Form A.

32. The method of claim 30, wherein the at least one crystalline form of 3-(2R-tetrahydrofuryl-methyl)-2-thioxanthine consists essentially of 3-(2R-tetrahydrofuryl-methyl)-2-thioxanthine Form B.

33. The method of claim 30, wherein the at least one crystalline form is 3-(2R-tetrahydrofuryl-methyl)-2-thioxanthine Form A.

34. The method of claim 33, wherein the at least one crystalline form is in a substantially pure form.

35. The method of claim 30, wherein the at least one crystalline form is 3-(2R-tetrahydrofuryl-methyl)-2-thioxanthine Form B.

36. The method of claim 35, wherein the at least one crystalline form is in a substantially pure form.

37. The method of claim 30, wherein said disease or condition is selected from neuroinflammatory disorders, cardio- and cerebrovascular atherosclerotic disorders, peripheral artery disease, heart failure, and respiratory disorders.

38. The method according to claim 30, wherein said disease or condition is selected from multiple sclerosis and Parkinson's disease.

39. A crystalline form of 3-(2R-tetrahydrofuryl-methyl)-2-thioxanthine comprising 3-(2R-tetrahydrofuryl-methyl)-2-thioxanthine Form A.

40. The crystalline form of claim 39 consisting essentially of 3-(2R-tetrahydrofuryl-methyl)-2-thioxanthine Form A.

41. The crystalline form of claim 39, wherein said crystalline form is substantially pure.

42. The crystalline form according to claim 39, wherein at least about 90 wt. % of said crystalline form comprises 3-(2R-tetrahydrofuryl-methyl)-2-thioxanthine Form A.

43. A crystalline form of 3-(2R-tetrahydrofuryl-methyl)-2-thioxanthine comprising 3-(2R-tetrahydrofuryl-methyl)-2-thioxanthine Form B.

44. The crystalline form of claim 43 consisting essentially of 3-(2R-tetrahydrofuryl-methyl)-2-thioxanthine Form B.

45. The crystalline form of claim 43, wherein said crystalline form is substantially pure.

46. The crystalline form according to claim 43, wherein at least about 90 wt. % of said crystalline form comprises 3-(2R-tetrahydrofuryl-methyl)-2-thioxanthine Form B.
